(19) United States
(12) Patent Application Publication
(10) Pub. No.: US 2021/0346613 A1
(43) Pub. Date: Nov. 11, 2021

(54) CONTROLLED DELIVERY DEVICE FOR TREATING CORONAVIRUS INFECTIONS AND METHODS THEREOF

(71) Applicants: Mahesh Kumar KHAITAN, Chandigarh (IN); Rohit KHAITAN, Chandigarh (IN)

(72) Inventors: Mahesh Kumar KHAITAN, Chandigarh (IN); Rohit KHAITAN, Chandigarh (IN)

(21) Appl. No.: 17/105,992

(22) Filed: Nov. 27, 2020

(30) Foreign Application Priority Data
May 5, 2020 (IN) 202011019069

(51) Int. Cl. A61M 11/00; A61M 16/06; A61M 16/16; A61M 16/00; A61M 16/08; A61K 9/00; A61K 33/00; A61K 31/58

(52) U.S. Cl. CPC A61M 11/005; A61M 16/0666; A61M 16/06; A61M 16/16; A61M 2205/50; A61M 16/0833; A61K 9/0078; A61K 33/00; A61K 31/58; A61M 16/0057

(57) ABSTRACT

The present disclosure provides methods of treatment of COVID-19 through pH modulation of the respiratory tract using a controlled delivery device. In some embodiments, the controlled delivery device includes an aerosolizer, a microprocessor, and a syringe. The controlled delivery device can be integrated with a humidifier or a ventilator. In some embodiments, two agents are delivered in conjunction using the controlled delivery device. In some embodiments, one agent is a physiologically compatible alkalizing agent, such as an aqueous solution of sodium bicarbonate of strength 3%-8.4% at a pH ranging from 8 to 8.6 at 20° C. In some embodiments, one agent is an anti-inflammatory agent, such as corticosteroid ciclesonide.

### CROSS-REFERENCE TO RELATED APPLICATION

This application claims the benefit of priority of Indian Patent Application No. 202011019069, filed May 5, 2020, which is incorporated herein by reference in its entirety.

### TECHNICAL FIELD

The present disclosure relates to medical devices and methods for therapeutic use. More specifically, the present disclosure relates to integrable microprocessor controlled delivery devices for treating respiratory viral infections, including COVID-19, and methods of use.

### BACKGROUND

### COVID-19 and its Impact on Mankind

COVID-19 has subsumed the very existence of mankind and for want of a cure and locked down the entire world causing financial meltdown and complete uncertainty. During the first 3 weeks of the COVID-19 outbreak in the Seattle area, the most common reasons for admission to the ICU were hypoxemic respiratory failure leading to mechanical ventilation, hypotension requiring vasopressor treatment, or both. Mortality among these critically ill patients was high. Of the 1591 critically ill patients with laboratory-confirmed COVID-19 admitted to ICUs in Lombardy, Italy, a large proportion required mechanical ventilation and respiratory support involving high levels of positive end-expiratory pressure (PEEP), and ICU mortality was 26%. Worldwide, there are currently 3,320,613 confirmed cases and 234,393 deaths from the coronavirus COVID-19 outbreak as of May 1, 2020, as given in Table 1 below:

TABLE 1
Global Status of COVID-19

| CURRENTLY INFECTED | CASES WITH OUTCOME |
|---|---|
| 3,320,613 | 1,283,653 |
| In Mild Condition | Recovered/Discharged |
| 1,986,062 | 1,049,260 |
| Serious or Critical | Deaths |
| 50,898 | 234,393 |

Source: https://www.worldometers.info/coronavirus/

Current Approaches to Treatment of COVID-19 and their Limitations

Re-purposing of drugs—Detailed clinical trials are required to either identify promising therapeutic drugs or repurposed drugs that may alleviate the symptoms and accelerate recovery. Trials with hydroxychloroquine or chloroquine, which is an oral drug established for treating malaria, and Remdesivir, which is an intravenous injectable drug made for treating Ebola virus are ongoing. Remdesivir has been granted emergency FDA approval as it has shown reduction in mortality rate and reduction in recovery duration in some patients. Also, the ongoing efforts towards re-purposing of drugs is aimed at the 5-15% of the patients infected with COVID-19 who suffer from sever or mild symptoms. While several drug trials are ongoing, there are currently no drugs licensed for the treatment or prevention of COVID-19. If a patient has mild COVID-19 symptoms, a doctor typically recommends the patient to use some supportive care to relieve symptoms, such as pain relievers, rest, fluidic intake, and to recover at home (https://www.mayoclinic.org/diseases-conditions/coronavirus/diagnosis-treatment/drc-20479976).

Vaccines—Many companies in different countries are developing vaccines for COVID-19. 82 vaccines are in preclinical evaluation as of Apr. 26, 2020. (https://www.who.int/who-documents-detail/draft-landscape-of-covid-19-candidate-vaccines). Vaccines are mainly preventive and not therapeutic. The lag period and final approval of a new vaccine will take time. For example, it could take at least one and half to two years before a vaccine is ready for public use.

### SUMMARY

According to an exemplary embodiment of the present disclosure, a method of treating a subject having COVID-19 is provided. In some embodiments, the method includes delivering a first agent to a subject for a treatment session. In some embodiments, the treatment session includes receiving a quantity of the first agent by an aerosolizer, the first agent comprising 3%-8.4% sodium bicarbonate at a pH ranging from 8.0 to 8.6 at 20° C. In some embodiments, the treatment session also includes converting the first agent into aerosolized form. In some embodiments, the aerosolized form of the first agent includes droplets having an average size of 3 μm. In some embodiments, the treatment session further includes delivering the first agent in the aerosolized form to the subject via a face mask configured to cover the mouth and nose of the subject. In some embodiments, the quantity of the first agent is from 3 ml to 6 ml. In some embodiments, the treatment session has a treatment duration from 7 minutes to 30 minutes.

In some embodiments, the method further includes delivering the first agent in the aerosolized form with an air flow generated by a humidifier or a ventilator to the face mask. In some embodiments, the method includes repeating the treatment session after a gap period of 8 hours. In some embodiments, the method includes delivering the first agent for the treatment session daily for 14 to 30 days. In some embodiments, the method includes prophylactically treating a subject not having COVID-19 symptoms. In some embodiments, the method further includes capturing virus particles in exhaled air from the subject using a filter fitted in or attached to the face mask, wherein the filter is a N95 filter.

In some embodiments, the method includes delivering a second agent to the subject before delivering the first agent as a part of the treatment session. In some embodiments, the second agent is an anti-inflammatory agent. In some embodiments, the method includes delivering the second agent to the subject via the face mask. In some embodiments, the method includes delivering the second agent to the subject via a nasal cannula. In some embodiments, the anti-inflammatory agent is ciclesonide. In some embodiments, the method includes preparing a hypotonic aqueous suspension of ciclesonide, converting the hypotonic aqueous suspension into aerosolized form, and delivering the hypotonic aqueous suspension in the aerosolized form to the subject. In some embodiments, the method includes delivering the anti-inflammatory agent in a quantity ranging from 160 micrograms to 200 micrograms.

In some embodiments, the method includes using a controlled delivery device. In some embodiments, the controlled delivery device includes the aerosolizer and a controller. The controller includes a microprocessor and is operatively connected to the aerosolizer. In some embodiments, the aerosolizer includes a medication cup configured to contain the first agent. In some embodiments, the aerosolizer includes a vibratory mesh pad. In some embodiments, the aerosolizer includes a piezoelectric actuator. In some embodiments, the controlled delivery device includes a built-in timer circuit configured to set an operation period of the aerosolizer, and the controller is configured to switch off power supply to the aerosolizer when the set operation period is over. In some embodiments, the controlled delivery device further includes a syringe configured to deliver the quantity of the first agent into the aerosolizer. In some embodiments, the controlled delivery device further includes a tubing and one or more connectors providing a fluid path between an outlet of the syringe to an opening of the medication cup. In some embodiments, the tubing is configured to deliver the first agent from the syringe onto the vibrating mesh pad of the aerosolizer. In some embodiments, the controlled delivery device further includes a syringe pump configured to deliver the first agent onto the vibrating mesh pad of the aerosolizer at a predetermined delivery rate. In some embodiments, the controlled delivery device is configured to continuously operate the aerosolizer during the delivery of the first agent onto the vibrating mesh pad of the aerosolizer. In some embodiments, the controlled delivery device is integrable with a humidifier or a ventilator via a T-joint connector.

According to an exemplary embodiment of the present disclosure, a method of delivering an agent to a subject having COVID-19 is provided. In some embodiments, the method includes delivering a first agent to a subject for a treatment session. In some embodiments, the treatment session includes receiving a quantity of the first agent by an aerosolizer. In some embodiments, the first agent includes 3%-8.4% sodium bicarbonate at a pH ranging from 8.0 to 8.6 at 20° C. In some embodiments, the treatment session also includes converting the first agent into aerosolized form. In some embodiments, the aerosolized form of the first agent includes droplets having an average size of 3 μm. In some embodiments, the treatment session further includes delivering the first agent in the aerosolized form to the subject via a face mask configured to cover the mouth and nose of the subject. In some embodiments, the quantity of the first agent is from 3 ml to 6 ml. In some embodiments, the treatment session has a treatment duration from 7 minutes to 30 minutes.

According to an exemplary embodiment of the present disclosure, a kit for delivering an agent to a subject to prevent or relieve COVID-19 symptoms is provided. In some embodiments, the subject has COVID-19 symptoms. In some embodiments, the subject is tested positive for COVID-19 but is asymptomatic and/or has mild COVID-19 symptoms. In some embodiments, the subject does not have COVID-19 symptoms or is tested negative for COVID-19. The kit may be used by the subject to prevent or relieve COVID-19 symptoms. In some embodiments, the kit includes a syringe, an aerosolize, a controller, a face mask, and an agent. In some embodiments, the aerosolizer includes a vibratory mesh pad and a medication cup or chamber. In some embodiments, the controller includes a microprocessor and is operatively connected with the aerosolizer. In some embodiments, the face mask is configured to cover the mouth and nose of the subject. In some embodiments, the agent is to be delivered to the subject in aerosolized form. In some embodiments, the agent includes 3%-8.4% sodium bicarbonate at a pH ranging from 8.0 to 8.6 at 20° C.

According to an exemplary embodiment of the present disclosure, a kit for delivering an agent to a subject to prevent the onset of COVID-19 symptoms is provided. In some embodiments, the agent is delivered to the subject as a prophylactic. In some embodiments, the kit includes a pressurized canister and a face mask. In some embodiments, the pressurized canister includes a valve and an opening. In some embodiments, the face mask is removably or fixedly attached to the pressurized canister at the opening. In some embodiments, the face mask is configured to cover the nose and mouth of a subject. In some embodiments, the pressurized canister contains an agent. In some embodiments, the agent includes 3%-8.4% sodium bicarbonate at a pH ranging from 8.0 to 8.6 at 20° C. In some embodiments, upon actuation of the valve, the agent exits the pressurized canister from the opening in aerosolized form into the face mask.

In some embodiments, the present disclosure provides controlled delivery devices for treating a respiratory viral infection. In some embodiments, the present disclosure provides methods of use of the controlled delivery devices for treating a respiratory viral infection. In some embodiments, the present disclosure provides methods of treating a respiratory viral infection by use of sodium bicarbonate (NaHCO₃). In some embodiments, the present disclosure provides methods of treating a respiratory viral infection by use of corticosteroid ciclesonide in conjunction with sodium bicarbonate. In some embodiments, the respiratory viral infection is COVID-19.

Additional disclosure of the disclosed embodiments will be set forth in part in the description that follows. It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain exemplary principles of certain disclosed embodiments as set forth in the accompanying claims.

### DETAILED DESCRIPTION OF THE DRAWINGS

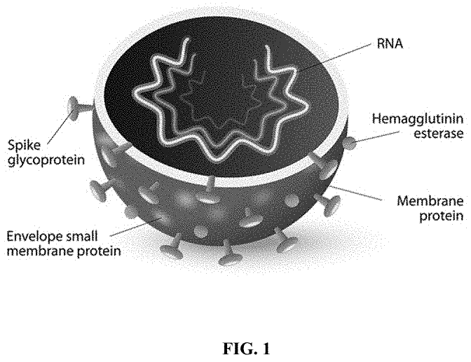

FIG. 1 illustrates a sectional view of the severe acute respiratory syndrome coronavirus 2 (SARS-CoV-2).

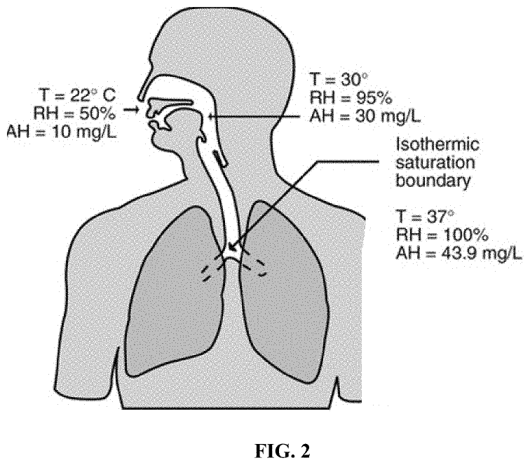

FIG. 2 illustrates the physiology of air entering the upper respiratory tract through the nose and mouth of a subject.

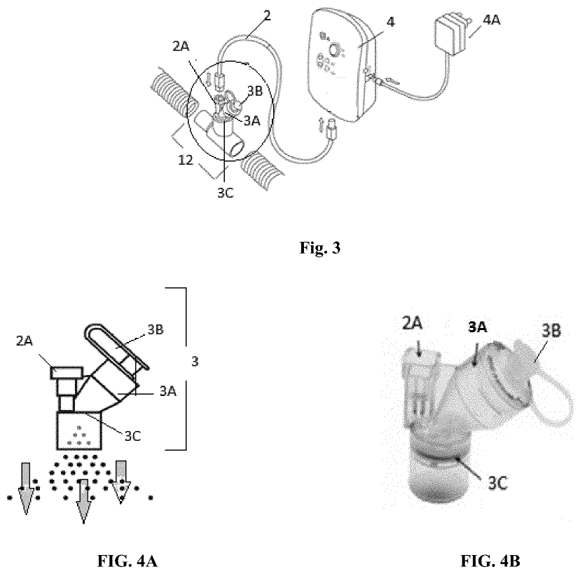

FIG. 3 illustrates an exemplary controlled delivery device, according to some embodiments of the present disclosure.

FIG. 4A illustrates an exemplary aerosolizer, according to some embodiments of the present disclosure.

FIG. 4B is a 3-D image of the exemplary aerosolizer of FIG. 4A.

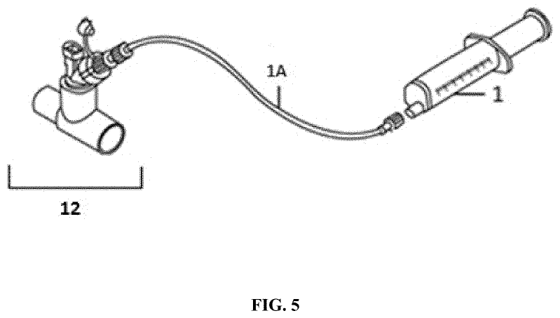

FIG. 5 illustrates delivery of an agent to an exemplary aerosolizer via a tubing connected to an exemplary syringe, according to some embodiments of the present disclosure.

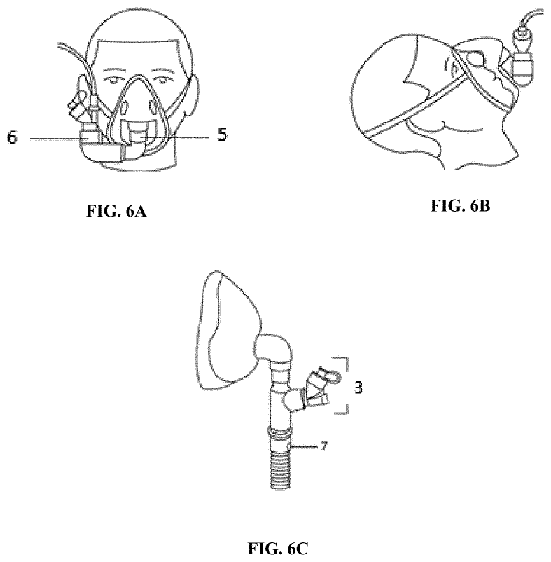

FIG. 6A illustrates delivery of aerosol to a subject through the mouth and nasal route using an exemplary face mask when the subject is in an upright position, according to some embodiments of the present disclosure.

FIG. 6B illustrates delivery of aerosol to a subject through the mouth and nasal route using an exemplary face mask when the subject is in a reclined position, according to some embodiments of the present disclosure.

FIG. 6C illustrates an exemplary aerosolizer connected with an exemplary face mask, according to some embodiments of the present disclosure.

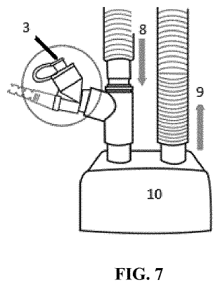

FIG. 7 illustrates the integration of an exemplary aerosolizer with a humidifier, according to some embodiments of the present disclosure.

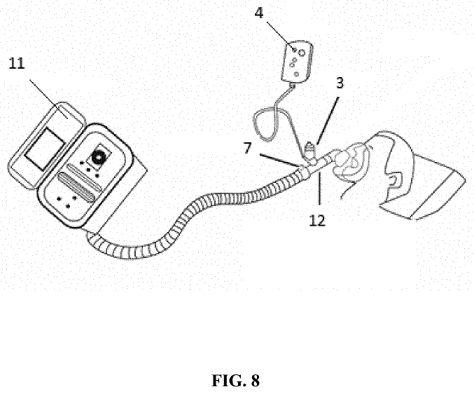

FIG. 8 illustrates the integration of an exemplary aerosolizer with a mechanical ventilator, according to some embodiments of the present disclosure.

### DETAILED DESCRIPTION

Vaccines for COVID-19 involve long lag periods and are mainly “preventive.” Recently, a study in India reported that COVID-19 antibodies may only last just 50 days, suggesting limited preventative effects of vaccines for COVID-19 (https://timesofindia.indiatimes.com/life-style/health-fitness/health-news/covid-antibodies-may-last-just-50-days/articleshow/77796532.cms). Re-purposed drugs for treating COVID-19 have major issues of toxicity at high doses and lack desired therapeutic efficacy. Indeed, while clinical trials of various re-purposed drugs are going on worldwide, there is still no specific antiviral treatment recommendation for COVID-19. The present disclosure provides a solution to the problem by providing an innovative approach for treating COVID-19 patients. In some embodiments, the present disclosure provides a therapeutic approach for treating COVID-19 by modulating the pH of the respiratory tract of a COVID-19 patient using a controlled delivery device. In some embodiments, the present disclosure provides methods of treatment that improve or relieve the symptoms of COVID-19 patients. In some embodiments, the present disclosure provides methods of treatment that reduce the mortality rate of COVID-19 patients. In some embodiments, the present disclosure provides methods of treatment of asymptomatic and/or mildly symptomatic COVID-19 patients. In some embodiments, the present disclosure provides methods of prophylactic treatment of a subject who does not have COVID-19 symptoms to protect the subject from COVID-19, to prevent the onset of COVID-19 symptoms, and/or to prevent the transmission of COVID-19. As described herein, although description of embodiments of the present disclosure uses COVID-19 as an example of respiratory viral infections, it is to be understood that embodiments of the present disclosure can equally be applied for treating other respiratory viral infections.

### Definitions

Integrable is defined as “capable of being integrated” (https://www.merriam-webster.com/dictionary/integrable); integrate is defined as to form, coordinate, or blend into a functional or unified whole or to seamlessly incorporate into a larger unit (https://www.merriam-webster.com/dictionary/integrate). Thus, as defined herein, the term “integrable” means that it can be integrated with another device or can work as a standalone device as well.

As defined herein, a “controlled delivery device” refers to a device that can deliver specific agents of embodiments of the present disclosure in aerosolized form to the respiratory tract, including the nasal passage, mouth, and lungs, of an infected patient in a controlled manner. In some embodiments, the device can be integrated in line with a ventilator or a humidifier such that the agent in aerosolized form is transported to the respiratory tract with the air flow generated by the ventilator or humidifier.

As defined herein, the term “agent” refers to refers to a chemical or synthetic substance that is administered to a subject to treat a disease, disorder, or other clinically recognized condition, or for prophylactic purposes, and has a clinically significant effect on the body of the subject to treat, prevent, and/or provide symptomatic relief from the disease, disorder, or condition.

As defined herein, “pH Modulation” refers to change of pH of the respiratory tract in controlled manner by use of a physiologically compatible alkalizing agent.

As defined herein, “respiratory tract” refers to the passage formed by the mouth, nose, throat, and lungs, through which air passes during breathing.

As defined herein, the “delivery of two or more agents in conjunction” refers to delivery of the two or more agents one after the other over a certain period of time, not simultaneously as one unit, for achieving improved therapeutic results, which are better than results achieved if agents are given alone.

As defined herein, COVID-19 refers to the disease caused by a specific mutated variety of coronavirus which has been assigned the name severe acute respiratory syndrome coronavirus 2 (SARS-CoV-2). SARS-CoV-2 refers to the mutated variety of coronavirus responsible for the COVID-19 disease (www.thelancet.com Vol 395 Mar. 21, 2020; weblink—https://www.thelancet.com/pdfs/journals/lancet/PIIS0140-6736(20)30557-2.pdf).

“Aerosolizer” is typically defined as “a device for aerosolizing” (https://en.wiktionary.org/wiki/aerosolizer; https://en.wiktionary.org/wiki/aerosoliser#English). “Aerosolize” is typically defined as “Convert into a fine spray or colloidal suspension in air” (https://www.lexico.com/en/definition/aerosolize). As defined herein, an “aerosolizer” in the present disclosure refers to a device for converting a liquid agent into aerosolized form (i.e., a fine spray of liquid or colloidal suspension in air) by a vibrating mesh pad. In some embodiments, the aerosolized form of the agent produced by the aerosolizer is delivered through a face mask to ensure inhalation through the nasal tract and mouth simultaneously by a subject.

A mechanical ventilator is a machine that helps a patient breathe (ventilate) when he or she cannot breathe on his or her own for any reason. Mechanical ventilation or assisted ventilation is the medical term for artificial ventilation where mechanical means are used to assist or replace spontaneous breathing. This may involve a machine called a ventilator, or the breathing may be assisted manually by a suitably qualified professional, such as an anesthesiologist, respiratory therapist (RT), registered nurse, or paramedic, by compressing a bag valve mask device.

As used herein, mechanical ventilation is invasive if it involves any instrument inside the trachea through the mouth, such as an endotracheal tube or the skin, such as a tracheostomy tube. Face or nasal masks are used for non-invasive ventilation in appropriately selected conscious patients (https://en.wikipedia.org/wiki/Mechanical ventilation).

As used herein, a vaccine is a biological preparation that provides active acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. Vaccines can be preventive or prophylactic (i.e., to prevent or ameliorate the effects of a future infection by a natural or “wild” pathogen), or therapeutic (e.g., vaccines against cancer, which are yet being investigated for therapeutic efficacy) (https://en.wikipedia.org/wiki/Vaccine).

As described herein, when a person receives active immunization, the vaccine prevents the infectious disease by activating the body's production of antibodies that can fight off invading bacteria or viruses. Passive immunization, in which antibodies against a particular infectious agent are given directly to the child or adult, is sometimes appropriate. (https://www.healthychildren.org/English/safety-prevention/immunizations/Pages/Immunizations %20Active-vs-Passive.aspx).

### Coronaviruses—A Category of RNA Viruses Responsible for Respiratory Infections

Coronaviruses are a large family, but only seven of its members infect humans. Four types cause minor illnesses like the common cold, while other coronaviruses have triggered far more devastating impacts, such as SARS, MERS, and now COVID-19. Coronaviruses account for up to 30 percent of upper respiratory tract infections in adults and are named “corona” (like a crown) because their membranes are studded by spike-like proteins. FIG. 1 illustrates a sectional view of the SARS-CoV-2. Epidemics in the past caused by coronaviruses and facts relating to the same including COVID-19 are given in Table 2 below:

TABLE 2
Disease Outbreaks Caused by Human Coronaviruses

| | SARS-CoV | MERS-CoV | SARS-CoV-2 |
|---|---|---|---|
| Disease Name | SARS | MERS | COVID-19 |
| | (Severe Acute | (Middle East | Corona Virus |
| | Respiratory | Respiratory | Disease |
| | Syndrome) | Syndrome) | |
| Year -Outbreak | 2002-2004 | 2012, 2015, 2018 | 2019-2020 |
| | | | Pandemic |
| Date- 1^{st} case | November, 2002 | June, 2012 | December, 2019 |
| Location | Shunde, China | Jeddah, Saudi | Wuhan, China |
| | | Arabia | |
| Age Average | 44 | 56 | 56 |
| Sex Ratio | 0.8:1 | 3.3:1 | 1.6:1 |
| (M/F) | | | |
| Confirmed | 8096 | 2494 | 1,783,941 |
| cases | | | |
| Deaths | 774 | 858 | 109,312 |
| Case Fatality | 9.2% | 37% | 6.1% |
| Rate | | | |

(Source- https://en.wikipedia.org/wiki/Coronavirus)

COVID-19 is a respiratory disease caused by SARS-CoV-2. It starts in the respiratory tract, causing pneumonia-like symptoms. Once the virus enters the human body, it looks for cell proteins called receptors. If the virus finds a compatible receptor, it can invade and start replicating itself. In March 2020, in the journal Science, a research team led by scientists at the University of Texas at Austin described the tiny molecular key on SARS-CoV-2 that gives the virus entry into the cell. This key is called a spike protein, or S-protein. (Daniel Wrapp et al. (2020) Cryo-EM structure of the 2019-nCoV spike in the prefusion conformation. Science. DOI: 10.1126/science.abb2507). The S-protein of SARS-CoV-2 has high affinities for human angiotensin-converting enzyme 2 (ACE2). ACE2 receptors are abundantly present in humans in the epithelia of the lung and small intestine, which might explain the most common route of entry for the COVID-19 being found in the lung cells. ACE2 receptors are also present in the other human organs, including oral and nasal mucosa, nasopharynx, oropharynx, hypopharynx, etc. The most common symptoms of COVID-19 include fever, cough, and dyspnea (i.e., difficult or labored breathing). These symptoms may appear 2 to 14 days after exposure.

The present disclosure provides a new approach for the treatment of COVID-19. In some embodiments, the new approach includes pH modulation through alkalization of the respiratory tract by using a controlled delivery device. In some embodiments, the controlled delivery device includes a microprocessor and a non-transitory computer readable medium that stores a set of instructions that is executable by the microprocessor. The set of instructions, when executed by the processor, allows the controlled delivery device to deliver one or more agents automatically without human intervention according to a predetermined session. In some embodiments, the microprocessor and a non-transitory computer readable medium are parts of a controller operatively connected with the controlled delivery device. In some embodiments, the controller connects with the controlled delivery device by a physical wire. In some embodiments, the controller connects with the controlled delivery device via a wireless network. In some embodiments, the controlled delivery device includes an aerosolizer configured to deliver one or more agents in aerosolized form. In some embodiments, the controlled delivery device is an integrable device that can be easily integrated with a humidifier or a ventilator for providing relief to patients. In some embodiments, the controlled delivery device is a standalone device that can be operated by a healthcare provider or the patient.

In some embodiments, the controlled delivery device includes a syringe configured to deliver an agent to the aerosolizer. In some embodiments, the agent is delivered to a medical cup or a chamber of the aerosolizer. In some embodiments, the controlled delivery device includes a tubing and one or more connectors providing a fluid path between an outlet of the syringe to an opening of the medication cup or chamber. In some embodiments, a tubing having a first connector at a first end and a second connector at a second end is used. In some embodiments, the first connector is a female luer connector that connects with a male luer connector at the outlet of the syringe. In some embodiments, the first connector is a non-standard luer connector to reduce risk of misconnection with other delivery systems. In some embodiments, the second connector is a luer-lock connector or a screw-lock connector that forms a fitting with a threaded connector at the opening of the medical cup or chamber of the aerosolizer.

In some embodiments, the syringe is filled with a suitable volume of the agent by a caregiver or a patient. In some embodiments, the syringe is prefilled with a suitable volume of the agent by a pharmacy or a manufacturer of the agent. In some embodiments, the suitable volume includes a predetermined volume of the agent to be delivered to the patient for one or more treatment sessions. In some embodiments, the suitable volume includes an additional volume of the agent for priming the syringe and/or its tubing and connectors before delivering the agent to the aerosolizer. In some embodiments, the syringe is manually operated to deliver a predetermined volume of the agent to the aerosolizer. In some embodiments, the syringe is connected with a syringe pump or a driver to automatically deliver a predetermined volume of the agent to the aerosolizer. In some embodiments, the pump rate of the syringe pump is adjusted in accordance with a predetermined delivery rate.

In some embodiments, the syringe pump operates continuously over a delivery period such that a predetermined amount of the agent is delivered to the aerosolizer in a drop-by-drop manner at a predetermined delivery rate during the delivery period. In some embodiments, the predetermined delivery rate of the syringe is in the unit of ml per hour and can be determined based on the predetermined volume (e.g., from 1 ml to 12 ml), delivery period (e.g., from 0.5 hour to 3 hours), and concentration of the agent (e.g., from 1 mg/ml to 100 mg/ml). For example, the predetermined delivery rate of the syringe can be calculated as: predetermined volume/period of delivery/concentration of the agent. In some embodiments, a predetermined volume of the agent is determined based on a desired dose of the agent for a treatment session and the concentration of the agent. For example, the predetermined volume of the agent for one treatment session can be calculated as: a single dose of the agent/concentration of the agent.

In some embodiments, the agent is delivered from the syringe via a tubing through the opening of the medication cup and dropped onto a vibrating mesh pad of the aerosolizer in a drop-by-drop manner. For example, the tubing may extend through the opening of the medication cup and towards the vibrating mesh pad such that a drop of the agent exiting the end of the tubing directly falls upon the vibrating mesh pad. In some embodiments, the aerosolizer operates continuously during the delivery period such that upon or immediately after a drop of the agent is delivered to the vibrating mesh pad, the drop of the agent is converted into aerosolized form to be delivered to the patient. In such cases, the delivery rate of the syringe determines the output rate of the aerosolizer. In some embodiments, the output rate of the aerosolizer equals the delivery rate of the syringe in the unit of ml per hour. In some embodiments, increasing the delivery rate of the syringe or the pump rate of the syringe pump increases the output rate of the aerosolizer, thereby increasing the rate of delivery of the agent in aerosolized form to the patient. In some embodiments, decreasing the delivery rate of the syringe or the pump rate of the syringe pump decreases the output rate of the aerosolizer, thereby decreasing the rate of delivery of the agent in aerosolized form to the patient. Advantageously, the drop-by-drop manner delivery of the agent to the vibration mesh pad of the aerosolizer allows accurate, efficient, and consistent delivery of a dose of the agent to the patient in a controlled manner. In some embodiments, the concentration of the agent in the aerosolized form does not change over the delivery period. In some embodiments, loss of the agent during delivery is reduced or minimized.

In some embodiments, a first agent is delivered by the controlled delivery device to a patient. In some embodiments, the first agent is a physiologically compatible alkalizing agent. In some embodiments, the alkalizing agent is sodium bicarbonate of a strength ranging from 3% to 8.4% (i.e., from 300 mg/10 mL to 840 mg/10 mL) at a pH ranging from 8.0 to 8.6 at 20° C. In some embodiments, sodium bicarbonate is delivered in a quantity ranging from 3 ml to 6 ml per session. In some embodiments, up to 2 sessions of delivery of sodium bicarbonate are performed. In some embodiments, two sessions of delivery of sodium bicarbonate with a gap of 8 hours between the two sessions are performed daily. In some embodiments, a session of delivery of sodium bicarbonate has a duration ranging from 7 minutes to 30 minutes. In some embodiments, sodium bicarbonate is delivered to a patient for a period ranging from 14 days to 30 days. The alkalizing agent leads to inactivation of the virus and prevents spread of the infectious disease. This is because the virus requires acidic environment (e.g., a pH ranging from 6.0 to 6.5) to attach and enter the cells and propagate. In some embodiments, the first agent includes one or more inactive ingredients or excipients. For example, the first agent may include water, nitrogen, edetate disodium, sodium hydroxide, or a selected combination thereof.

In some embodiments, a second agent is delivered by the controlled delivery device to a patient. In some embodiments, the second agent is an anti-inflammatory agent. In some embodiments, the second agent is corticosteroid ciclesonide, which is anti-inflammatory and prevents bronchospasm. In some embodiments, the second agent is delivered to the patient in aerosolized form. In some embodiments, the second agent is delivered in a quantity ranging from 160 micrograms to 200 micrograms. In some embodiments, a hypotonic aqueous suspension of ciclesonide is prepared, which contains microcrystalline cellulose, carboxymethylcellulose sodium, hypromellose, and potassium sorbate and edetate sodium. In some embodiments, the hypotonic aqueous suspension of ciclesonide includes hydrochloric acid to adjust the pH of the hypotonic aqueous suspension to 4.5.

In some embodiments, the first agent and the second agent are delivered in conjunction to a patient. In some embodiments, the first agent is delivered after the second agent. In some embodiments, a gap period between the delivery of the two agents is from 2 to 5 hours. The two agents are delivered in conjunction and not simultaneously as one unit is due to the instability of ciclesonide hypotonic aqueous suspension at alkaline pH (See European Patent No. 1 697 398 B1). In some embodiments, the delivery of the first agent and the second agent in conjunction reduces or prevents bronchospasm and inflammation, thereby providing significant and quick relief to the patient.

In some embodiments, one or more agents is provided in an ampule. For example, when the first agent is sodium bicarbonate, a fixed volume of 3% to 8.4% sodium bicarbonate having a pH ranging from 8.0 to 8.6 at 20° C. is provided in an ampule. when the second agent is ciclesonide, a fixed volume of hypotonic aqueous suspension of ciclesonide is provided in an ampule. In some embodiments, one or more agents are loaded into the controlled delivery device in a fixed quantity manually or automatically. In some embodiments, one or more agents are withdrawn from an ampule and loaded into the controlled delivery device manually. In some embodiments, one or more agents are withdrawn from an ampule into a syringe. In some embodiments, one or more agents are loaded into the controlled delivery device in a controlled manner by a mechanized depression of the plunger of a syringe. In some embodiments, the aerosolizer of the controlled delivery device includes a vibrating mesh pad that aerosolizes the one or more agents loaded into the controlled delivery device such that the one or more agents are delivered in aerosolized form to the patient. In some embodiments, the one or more agents are delivered to the respiratory tract of the patient. In some embodiments, the one or more agents are delivered to the nasal passages, mouth, and lungs via a face mask. In some embodiments, the face mask is a non-invasive ventilator face mask or a ventilated nebulizer mask configured to be placed over the mouth and nose of the patient receiving the treatment. In some embodiments, the face mask includes one or more suitable filters attached (e.g., N95 or above filter) to capture the virus particles in the exhaled air. In some embodiments, a nasal canula is used for delivery of aerosolized ciclesonide.

It is advantageous to deliver the aerosolized alkalizing agent via a face mask because the face mask facilitates alkalization in the nose, mouth, pharynx, and the lungs simultaneously, which in turn can achieve higher or complete deactivation of the viruses, e.g., SARS-CoV-2, in the respiratory tract as these are the areas where the ACE2 receptors are found in the respiratory tract to which the virus attaches for entry and spread.

In some embodiments, the viral proteins responsible for the attachment and entry of the viruses are themselves inactivated by the pH modulation of the respiratory tract achieved by the controlled delivery of the alkalizing agent to the tract using the controlled delivery device. In some embodiments, the delivery of the alkalizing agent to the tract using the controlled delivery device effectively treat COVID-19 by reducing or eliminating one or more symptoms of the patient.

In some embodiments, in case of asymptomatic patients tested positive for COVID-19 (e.g., as assessed by RT-PCR diagnostic test), the alkalizing agent is initially delivered to the patient using the controlled delivery device. In some embodiments, when the patient experiences any bronchospasms, the anti-inflammatory agent is delivered in conjunction to the patient using the controlled delivery device.

A standard protocol for an infectious disease like COVID-19 is that upon confirmation through RT-PCR Test, at least 14-15 days of isolation are required for patients who are asymptomatic but tested positive for COVID-19. Even longer periods of isolation are required for symptomatic patients until they are discharged from hospitals after recovery (i.e., tested negative for COVID-19). This protocol is presumed to be applicable at all times during the existence of this pandemic. Thus, in some embodiments, a therapeutic treatment of is provided that is applicable for the entire duration of the patient's isolation.

The patients infected with COVID-19 sometimes develop fever. In some embodiments, to alleviate fever, one or more suitable antipyretic drugs are administered to a patient in addition to the delivery of the alkalizing and anti-inflammatory agents.

In some embodiments, the present disclosure offers a practical and easy therapeutic approach to treat patients suffering from respiratory viral infectious diseases, such as COVID-19, airways reactive dysfunction syndrome (ARDS), or severe acute respiratory syndrome (SARS). The therapeutic approach is fast, inexpensive, and easy to scale up. Advantageously, embodiments of the present disclosure reduces mortality rate and markedly improve symptoms of discomfort in COVID-19 patients.

### Scientific Rationale

Acidification of cellular environment in the respiratory tract and lungs is a key factor in coronavirus attachment and spread. The internalization of the virus into the nucleus of the cell is dependent on a low pH in the cellular environment. The trafficking and processing steps that occur in cells that are infected with a coronavirus play a crucial role in the outcome of infection.

The spike glycoprotein (S protein) on the virion surface mediates receptor recognition and membrane fusion. During viral infection, the trimeric S protein is cleaved into S1 and S2 subunits and S1 subunits are released in the transition to the post fusion conformation. S1 contains the receptor binding domain (RBD), which directly binds to the peptidase domain (PD) of angiotensin-converting enzyme 2 (ACE2), whereas S2 is responsible for membrane fusion. When S1 binds to the host receptor ACE2, another cleavage site on S2 is exposed and is cleaved by host proteases, a process that is critical for viral infection. (Alexandra et al. (2020) Structure, function and antigenicity of the SARS-CoV-2 spike glycoprotein; doi: https://doi.org/10.1101/2020.02.19.956581). Following replication, the newly formed viral genomes leave the nucleus and assemble into infectious particles at the plasma membrane. The targeting and processing of the various viral components at this stage of the infectious cycle can have a major effect on the ability of the virus to spread and cause disease in its host.

Coronaviruses are activated at acidic pH, e.g. at pH 6.0. A study by Sturman et al. in 1990 (Sturman et al. (1990) Conformational change of the coronavirus peplomer glycoprotein at pH 8.0 and 37 degrees C. correlates with virus aggregation and virus-induced cell fusion. J Virol. 64(6):3042-3050) describes that coronavirus MHV-A59 (Coronavirus mouse hepatitis virus) is stable at pH 6.0 and 37 degree Celsius (half-life being approximately 24 hours) but is rapidly and irreversibly inactivated by brief treatment at pH 8.0 and 37 degree Celsius (half-life being approximately 30 minutes) using a particular buffer solution under in vitro conditions using cultured cell lines (17 Cl 1 and L2 lines of mouse fibroblasts). The importance of these changes of coronavirus MHV-A59 was reflected in the loss of virus infectivity, the aggregation of virions, and the increased virus-induced cell fusion at the same pH.

### Physiology of Air Entering the Respiratory Tract Through Nose and Mouth

In some embodiments of the present disclosure, the agents are delivered in aerosolized form and automatically reach the desired temperature of 37 degree Celsius on reaching the lungs, as depicted in FIG. 2. The nose heats and humidifies air on inhalation and cools and reclaims water from air that is exhaled. For example, as shown in FIG. 2, air having a relative humidity (RH) of 50% and an absolute humidity (AH) of 10 mg/L at 22° C. upon inhalation through the nose can have a RH of 95% and an AH of 30 mg/L at 30° C. at the pharynx and a RH of 100% and an AH of 43.9 mg/L at 37° C. at the lungs. When a person inhales air through the mouth at normal room temperature, pharyngeal temperatures are approximately 3° C. less than when the person breathes through the nose. Also, air inhaled through the mouth has 20% less RH than air inhaled through the nose. As shown in FIG. 2, as the inhaled air moves into the lungs, it achieves “BTPS” conditions (BT refers to body temperature, i.e. 37° C.; P refers to barometric pressure; S refers to saturated with water vapor, i.e., 100% relative humidity at 37° C.). Thus, in some embodiments, the aerosolized agents delivered by the controlled delivery device meet the BTPS conditions when reaching the lungs, including the desired temperature of 37° C., to inactivate the coronaviruses.

### Aerosolized Sodium Bicarbonate—Mechanisms and Pharmacological Safety Aspects

Sodium bicarbonate is a nonspecific antidote effective in the treatment of a variety of poisonings, including pulmonary injuries resulting from phosgene and chlorine gas inhalation exposures. Inhaled sodium bicarbonate neutralizes the hydrochloric acid that is formed when phosgene or chlorine gas react with the water in the respiratory tree. Inhalation of 5 mL of 5% sodium bicarbonate solution (prepared by mixing 3 mL of 8.4% sodium bicarbonate with 2 mL of normal saline) has been reported (Bosse G M (1994). Nebulized sodium bicarbonate in the treatment of chlorine gas inhalation. J Toxicol Clin Toxicol. 32(3):233-41). Some literature relating to safety of inhaled sodium bicarbonate solution is summarized in Table 3.

TABLE 3
Quantity and Concentrations of Sodium Bicarbonate solutions used for nebulization

| S. | Qty. | | | |
|---|---|---|---|---|
| No. | used | | Concentration | Reference |
| 1. | 4 | cc | 3.75% | Vinsel P J (1990) Treatment of acute chlorine gas inhalation |
| | | | | with nebulized sodium bicarbonate. J Emerg Med 8(3): 327- |
| | | | | 329. |
| 2. | 4.25 | ml | 3.75% | Douidar S M (1997) Nebulized sodium bicarbonate in acute |
| | | | | chlorine inhalation. Pediatr Emerg Care 13(6): 406-407. |
| 3. | — | | 8.4% | Hurlbut K et al. (1992) Pharmacotherapy of hazardous |
| | | | | materials toxicity. In: Sullivan J B, Krieger G R (eds) |
| | | | | Hazardous materials toxicology: clinical principles of |
| | | | | environmental health. Publisher: Williams & Wilkins, |
| | | | | Baltimore, pp. 405-414. |
| 4. | 5 | ml | 5% | Bosse G M (1994) Nebulized sodium bicarbonate in the |
| | | | | treatment of chlorine gas inhalation. J Toxicol Clin Toxicol |
| | | | | 32(3): 233-41. |
| 5. | 4 | ml | 3.75-5% | Howard C et al. (2007) Management of chlorine gas |
| | | | | exposure. J Emerg Nurs. 33: 402-4. |
| 6. | 4 | ml | 4.20% | Aslan et al. (2006) The Effect of Nebulized NaHCO3 |
| | | | | Treatment on “RADS” Due to Chlorine Gas Inhalation. |
| | | | | Toxicology, 18: 895-900. |
| 7. | 50 | ml | 8.4% | El Badrawy et al. (2018) Effect of sodium bicarbonate 8.4% |
| | | | | on respiratory tract pathogens. Chest Lung Res 1(1): 3-7. |
| 8. | 5 | ml | 4.2-8.4% | Carla Cristina Souza Gomez et al. (2019) Safety, |
| | | | | Tolerability, and Effects of Sodium Bicarbonate Inhalation |
| | | | | in Cystic Fibrosis, Clinical Drug Investigation 40, 105-117. |
| | | | | https://doi.org/10.1007/s40261-019-00861-x |

Thus, safety of the use of 3% to 8.4% sodium bicarbonate solution at a pH ranging from 8.0 to 8.6 at 20° C. in a quantity ranging from 3 ml to 6 ml as described in some embodiments of the present disclosure is well-supported by clinical use and related case studies and publications.

Ciclesonide belongs to a class of medications called corticosteroids. It works by decreasing swelling and irritation in the airways to allow for easier breathing. (https://medlineplus.gov/druginfo/meds/a609004.html). The therapeutic action of ciclesonide is achieved after the inhaled parent compound (CIC) is cleaved by esterases in the lungs to its active metabolite (des-CIC), a corticosteroid with high receptor affinity and anti-inflammatory activity. Safety aspects of inhaling ciclesonide have been reviewed (T. J. Schaffner. et al. (2009) Ciclesonide: a safe and effective inhaled corticosteroid for the treatment of asthma. Journal of Asthma and Allergy, 2:25-32. https://doi.org/10.2147/jaa.s4651). Ciclesonide is one of the safest corticosteroids, free from the side-effects associated with other typical steroidal drugs. In some embodiments, a single dose of aerosolized ciclesonide including from 160 micrograms to 200 micrograms of ciclesonide is delivered to the patient. Ciclesonide nasal spray works by preventing and decreasing inflammation (i.e., swelling that can cause other symptoms) in the trachea. (https://medlineplus.gov/druginfo/meds/a607008.html). Nebulized ciclesonide formulation has been reported for in vivo inhalation (T. Fu et al. (2018) Ciclesonide and Budesonide Suspensions for Nebulization Delivery: An In Vivo Inhalation Biopharmaceutics Investigation, International Journal of Pharmaceutics, 549(1-2): 21-30. doi: https://doi.org/10.1016/j.ijpharm.2018.07.048).

### PRIOR ART

U.S. Pat. No. 8,858,917 B2 is titled “Methods for limiting spread of pulmonary infections.” This patent describes formulations for pulmonary administration including a material that alters physical properties such as surface tension and surface elasticity of lung mucus lining fluid, which may be a surfactant and optionally, a carrier. These may include proteins such as albumin or polysaccharides such as dextran, which also has surface active properties, or polymers such as polyethylene oxide (PEO) or biodegradable synthetic polymers which can be used to encapsulate or deliver the materials to be delivered. The materials are disclosed for pulmonary administration by using a dry powder inhaler or metered dose inhaler.

US 2008/0000473 describes pH-based methods and devices for preventing hemagglutinin cell membrane fusion, e.g. in case of infections caused by influenza viruses. However, mode of infection of coronaviruses is totally different from influenza viruses. Spike proteins present in coronaviruses which are responsible for attachment of the virus to ACE2 receptors are not present in influenza viruses at all.

WO2014066856A1 is titled “Inhalable influenza vaccine compositions and methods.” This publication describes dry powder inhalable compositions and methods for using and making the compositions for vaccinating a subject against disease.

None of the prior art discussed above discloses the innovative approach for treating COVID-19 provided by the present disclosure. The innovative approach of the present disclosure allows for inactivation of SARS-Cov-2 virus by pH modulation through alkalization of the respiratory tract.

The World Health Organization (W.H.O) provides a database of literature reporting treatments and research of COVID-19. As of Apr. 29, 2020, a keyword search for “Sodium Bicarbonate” returns no results. A keyword search for “nebulizer” returns 4 documents, of which 2 relate to the possible propagation of the virus from the patient receiving nebulization, one for a distinct drug as a proposed therapy and one that concludes “For patients who need aerosol therapy, dry powder inhaler metered dose inhaler with spacer is recommended for spontaneous breathing patients; while vibrating mesh nebulizer is recommended for ventilated patients and additional filter is recommended to be placed at the expiratory port of ventilation during nebulization.” (DOI: 10.3760/cmaiissn.1001-0939.2020.0020) (https://pubmed.ncbi.nlm.nih.gov/32077661/). The web address of the database is “https://search.bvsalud.org/global-literature-on-novel-coronavirus-2019-ncov/?output=site&lang=en&from=0&sort=&format=summary&count=20&fb=&page=l&s kfp=&index=tw&q=&search_form_submit=” In addition, a recent clinical trial posted on Apr. 1, 2020, proposes the use of aerosol ciclesonide with hydroxychloroquine. In this clinical trial proposal, there is no mention at all of using aerosol of ciclesonide in conjunction with sodium bicarbonate in aerosolized form for cellular alkalization to inactivate the COVID-19 virus. So far, no researchers or studies contemplated the innovative approach for treating COVID-19 as provided by the present disclosure.

As described above, the present invention discloses a new approach for the treatment of COVID-19 disease by pH modulation through alkalization of the respiratory tract using a controlled delivery device. Exemplary embodiments of the controlled delivery device are described below with reference to the figures.

FIG. 3 illustrates an exemplary controlled delivery device, according to some embodiments of the present disclosure. FIG. 4A illustrates an exemplary aerosolizer, according to some embodiments of the present disclosure. FIG. 4B is a 3-D image of the exemplary aerosolizer of FIG. 4A. In some embodiments, as shown in FIGS. 3, 4A, and 4B, the controlled delivery device includes an aerosolizer (3) and a controller (4). The aerosolizer (3) aerosolizes and delivers one or more agents in aerosolized form. In some embodiments, the controller (4) includes a microprocessor and a non-transitory computer readable medium (not shown). In some embodiments, as shown in FIG. 3, the controller (4) is operatively connected with the aerosolizer (3) by a physical wire. For example, the aerosolizer (3) includes a socket (2A) for connecting to a USB power cable (2). In some embodiments, the USB power cable (2) is connected to the socket (2A) at a first connector and connected to a socket (not shown) in the controller (4) at a second connector. In some embodiments, the controller connects with the controlled delivery device via a wireless network. The controller (4) automatically controls delivery of one or more agents without human intervention.

In some embodiments, the controller (4) includes a built-in timer circuit configured to set an operation period of the aerosolizer (3). In some embodiments, the built-in timer circuit operates in two mode. In some embodiments, in a first mode, the controller (4) switches off power supply to the aerosolizer (3) when the set operation period is over. In such cases, the operation period equals to or is longer than the delivery period of the syringe. In some embodiments, in a second mode, the aerosolizer (3) operates continuously until it is manually switched off. In some embodiments, the controller (4) is powered by a universal adapter (4A) configured to be connected to a power outlet. In some embodiments, the controller (4) is powered by one or more batteries. In some embodiments, the aerosolizer (3) receives power from the controller (4) through a physical wire or a wireless connection.

As shown in FIGS. 4A and 4B, in some embodiments, the aerosolizer (3) includes a medication cup or chamber (3A). In some embodiments, the medication cup or chamber (3A) is a cavity that can receive a quantity of an agent in liquid form. In some embodiments, the medication cup or chamber (3A) can contain a quantity of the agent in liquid form ranging from 3 ml to 6 ml. In some embodiments, after receiving a quantity of the agent, the opening of the medication cup or chamber (3A) is sealed by a plug (3B). In some embodiments, the plug (3B) is a silicone plug. In some embodiments, the aerosolizer (3) includes a vibratory mesh pad (3C). In some embodiments, the vibratory mesh pad (3C) includes a plurality of perforations of a size ranging from 1 μm to 5 μm. In some embodiments, the plurality of perforations has an average size of 3 μm. In some embodiments, the vibratory mesh pad (3C) is made of an inert noble metal. In some embodiments, the aerosolizer (3) includes a piezoelectric actuator that vibrates the vibratory mesh pad (3C) at a high frequency to convert the liquid agent in the medication cup or chamber (3A) into aerosolized form. In some embodiments, the liquid agent is aerosolized into fine droplets of a size ranging from 1 μm to 5 μm with an average size of 3 μm. In some embodiments, the agent then exits the controlled delivery device in the aerosolized form from a delivery port of the aerosolizer (3).

FIG. 5 illustrates delivery of an agent to an exemplary aerosolizer via a tubing (1A) connected to an exemplary syringe (1), according to some embodiments of the present disclosure. In some embodiments, the syringe (1) is a variable syringe. In some embodiments, the syringe (1) includes a pump or a driver to deliver an agent to the medication cup or chamber (3A) of the aerosolizer (3). As shown in FIG. 5, in some embodiments, the plug (3B) is removed and a tubing (1A) is connected to the opening for the medication cup or chamber (3A) at a first end and connected to the syringe (1) at a second end. The medication cup or chamber (3A) receives the agent from the syringe (1) via the tubing (1A).

In some embodiments, the aerosolized agent exiting from a delivery port (6) of the aerosolizer (3) directly passes to a face mask. In some embodiments, as shown in FIG. 5, a T-joint adapter (12) is connected to the aerosolizer (3). The T-joint adapter (12) allows the aerosolized agent to be entrained with an air flow towards a face mask. In some embodiments, the air flow is generated by a humidifier. In some embodiments, the air flow is generated by a ventilator. In some embodiments, the aerosolizer (3) is integrated with a humidifier (10) via the T-joint adapter. For example, in some embodiments, as shown in FIG. 7, the aerosolizer (3) can be fitted on a tubing for leading air into the humidifier (10) at a suction end (8) via the T-joint adapter. In other embodiments, the aerosolizer (3) can be fitted on a tubing for leading air away from the humidifier (10) at the dry end (9) via the T-joint adapter. In some embodiments, the aerosolizer (3) is integrated with a mechanical ventilator (11) via the T-joint adapter, as shown in FIG. 8.

Exemplary methods of use of the controlled delivery device of the present disclosure are described below. In some embodiments, a method of use of the controlled delivery device include the following steps.

In some embodiments, an alkalizing agent is filled into the controlled delivery device. For example, the alkalizing agent is filled into the medication cup or chamber (3A) of the aerosolizer (3).

In some embodiments, as shown in FIGS. 6A and 6B, the delivery port (6) of the aerosolizer (3) is connected to a tubing leading to a face mask. In some embodiments, a universally compatible and integrable T-joint adaptor (12) as shown in FIG. 5 can be used to connect the delivery port (6) of the aerosolizer (3) to a tubing.

In some embodiments, a face mask is fit over the face of a patient to cover the mouth and nose. FIG. 6A illustrates delivery of aerosol to a subject through the mouth and nasal route using an exemplary face mask when the subject is in an upright position, according to some embodiments of the present disclosure. FIG. 6B illustrates delivery of aerosol to a subject through the mouth and nasal route using an exemplary face mask when the subject is in a reclined position, according to some embodiments of the present disclosure. In some embodiments, as shown in FIG. 6A, the face mask has a face mask elbow (5). In some embodiments, a filter, e.g. a N95 filter, is fitted at the face mask elbow (5) to capture the virus particles in the exhaled breath. In some embodiments, as shown in FIG. 6C, the tubing connecting the face mask for releasing air exhaled by the patient includes an exhalation port (7). In some embodiments, the exhalation port (7) is fitted with a filter, e.g. a N95 filter, to trap the virus particles in the exhaled air before the exhaled breath condensate is released.

In some embodiments, power is provided to the aerosolizer (3) to activate the vibratory mesh pad (3C). In some embodiments, the vibratory mesh pad (3C) vibrating at a high frequency converts the first agent, such as a physiologically compatible alkalizing agent, to aerosolized form and pumps the aerosolized agent via the face mask to the patient. In some embodiments, during operation of the aerosolizer (3), the vibratory mesh pad (3C) vibrates at a frequency ranging from 125 kHz to 135 kHz. In some embodiments, during operation of the aerosolizer (3), the vibratory mesh pad (3C) vibrates at a frequency at or about 128 kHz.

In some embodiments, a second agent, such as an anti-inflammatory agent, is delivered to the patient in conjunction with the first agent. In some embodiments, the second agent is delivered to the patient before the first agent. In some embodiments, the second agent is delivered in the same manner as the first agent by the aerosolizer (3). In some embodiments, the second agent is delivered after a gap period ranging from 2 to 5 hours. In some embodiments, the second agent is delivered via a face mask fitting over the mouth and nose of the patient. In some embodiments, the second agent is delivered via a nasal canula.

### Exemplary Delivery Protocol of an Alkalizing Agent

In some embodiments, the controlled delivery device is used to deliver an alkalizing agent according to a delivery protocol. In some embodiments, when the alkalizing agent is sodium bicarbonate of a strength ranging from 3% to 8.4% at a pH ranging from 8.0 to 8.6 at 20° C., a delivery protocol includes the following:
i. Quantity per session—3 ml-6 ml
ii. Time duration of each session—7 minutes-30 minutes
iii. Sessions/day—2 sessions with a gap of 8 hours daily
iv. Duration of treatment—14-30 days

### Exemplary Delivery Protocol of an Anti-Inflammatory Agent

In some embodiments, the controlled delivery device is used to deliver an anti-inflammatory agent according to a delivery protocol. In some embodiments, the anti-inflammatory agent is ciclesonide. A delivery protocol of ciclesonide includes delivering a ciclesonide solution to the patient in aerosolized form in a quantity ranging from 160 micrograms to 200 micrograms per day. In some embodiments, the ciclesonide solution is to be filled into the aerosolizer (3) using the syringe (1) attached to the aerosolizer (3) to ensure an accurate metered dose of ciclesonide for patients with severe symptoms. This protocol allows for “non-contact” controlled delivery of the anti-inflammatory agent to a patient suffering from COVID-19 with reduced risk or without any risk of contamination. In some embodiments, the anti-inflammatory agent is delivered to the patient using a nasal canula to avoid irritation in the oral passage. In some embodiments, the alkalizing agent and the anti-inflammatory agent are delivered in conjunction, i.e. one after the other and not simultaneously as one unit.

### Exemplary Safety Features of the Controlled Delivery Device

Since the transmission of the virus is very high from the exhaled breath condensate (EBC) from the patient, in some embodiments, a suitable filter (e.g., a N95 or above filter) is fitted in the round ports of a nebulizer face mask or a suitable anti-viral filter is attached to the discharge port of the non-invasive face mask to capture the condensate before releasing the exhaled air. This is a critical control for the safety of the medics and health workers treating/assisting in the treatment and care of the patients affected with COVID-19. In some embodiments, the tube through which the exhaled breath is being discharged is further connected to an appropriate filter (e.g., a N95 or above filter) to capture any virus that may be present in the exhaled air.

In some embodiments, the controlled delivery device and the agents can be manufactured on industrial scale. In some embodiments, the controlled delivery device and the agents can be used for treatment of COVID-19 patients in hospitals and under critical care in isolation facilities. In some embodiments, the controlled delivery device and the agents can be used by a COVID-19 patient in isolation for treating mild symptoms or as a prophylactic treatment.

In some embodiments, a kit for delivering an agent to a subject having COVID-19 is provided. The kit may be used by the subject to prevent or relieve the symptoms of COVID-19. In some embodiments, the kit includes a syringe, an aerosolize, a controller, a face mask, and an agent. The aerosolizer includes one or more components as described above, such as a vibratory mesh pad and a medication cup or chamber. The controller includes a microprocessor and is operatively connected with the aerosolizer. The face mask is configured to cover the mouth and nose of the subject. The agent is to be delivered to the subject in aerosolized form. In some embodiments, the agent includes 3%-8.4% sodium bicarbonate at a pH ranging from 8.0 to 8.6 at 20° C.

In some embodiments, a kit for delivering an agent to a subject to prevent the onset of COVID-19 symptoms is provided. In some embodiments, the agent is delivered to the subject as a prophylactic. In some embodiments, the kit includes a pressurized canister and a face mask. In some embodiments, the pressurized canister is a part of a metered dose inhaler. The pressurized canister includes a valve and an opening. The face mask is removably or fixedly attached to the pressurized canister at the opening. The face mask is configured to cover the nose and mouth of a subject. The pressurized canister contains an agent. In some embodiments, the agent includes 3%-8.4% sodium bicarbonate at a pH ranging from 8.0 to 8.6 at 20° C. Upon actuation of the valve, the agent exits the pressurized canister from the opening in aerosolized form into the face mask. The aerosolized agent is delivered to the subject via the face mask.

### NUMBERING OF PARTS

1—Syringe for delivery of metered quantity of agents to Aerosolizer
1A—Syringe Tubing
2—USB power cable
2A—Socket for USB power cable
3—Aerosolizer
3A—Medication cup or chamber
3B—Plug
3C—Vibratory Mesh Pad
4—Controller
4A—Universal Power Adaptor
5—Face Mask Elbow
6—Delivery Port
7—Exhalation Port
8—Tubing for leading air into the humidifier
9—Tubing for leading air away from the humidifier
10—Humidifier
11—Mechanical Ventilator
12—T-Joint adapter to connect Aerosolizer sequentially with ventilator and/or humidifier

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. Moreover, while illustrative embodiments have been described herein, the scope of the disclosure includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive.

It is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as exemplary only, with a true scope and spirit of the disclosed embodiments being indicated by the following claims.

What is claimed is:

1. A method of treating a subject having COVID-19, the method comprising:
delivering a first agent to a subject for a treatment session comprising:
receiving a quantity of the first agent by an aerosolizer, the first agent comprising 3%-8.4% sodium bicarbonate at a pH ranging from 8.0 to 8.6 at 20° C.;
converting the first agent into aerosolized form, the aerosolized form of the first agent comprising droplets having an average size of 3 μm; and
delivering the first agent in the aerosolized form to the subject via a face mask configured to cover the mouth and nose of the subject;
wherein the quantity of the first agent is from 3 ml to 6 ml; and
wherein the treatment session has a treatment duration from 7 minutes to 30 minutes.

2. The method of claim 1, further comprising delivering the first agent in the aerosolized form with an air flow generated by a humidifier or a ventilator to the face mask.

3. The method of claim 1, further comprising repeating the treatment session after a gap period of 8 hours.

4. The method of claim 1, further comprising delivering the first agent for the treatment session daily for 14 to 30 days.

5. The method of claim 1, further comprising prophylactically treating a subject not having COVID-19 symptoms.

6. The method of claim 1, further comprising delivering a second agent to the subject before delivering the first agent as a part of the treatment session, the second agent being an anti-inflammatory agent.

7. The method of claim 6, further comprising delivering the second agent to the subject via the face mask.

8. The method of claim 6, further comprising delivering the second agent to the subject via a nasal cannula.

9. The method of claim 1, wherein the anti-inflammatory agent is ciclesonide.

10. The method of claim 9, further comprising
preparing a hypotonic aqueous suspension of ciclesonide;
converting the hypotonic aqueous suspension into aerosolized form; and
delivering the hypotonic aqueous suspension in the aerosolized form to the subject.

11. The method of claim 9, further comprising delivering the anti-inflammatory agent in a quantity ranging from 160 micrograms to 200 micrograms.

12. The method of claim 1, further comprising capturing virus particles in exhaled air from the subject using a filter fitted in or attached to the face mask, wherein the filter is a N95 filter.

13. The method of claim 1, further comprising using a controlled delivery device, the controlled delivery device comprising:
the aerosolizer; and
a controller comprising a microprocessor, the controller being operatively connected to the aerosolizer;
wherein the aerosolizer comprises
a medication cup configured to contain the first agent;
a vibratory mesh pad; and
a piezoelectric actuator.

14. The method of claim 13, wherein the controller comprises a built-in timer circuit configured to set an operation period of the aerosolizer, and the controller is configured to switch off power supply to the aerosolizer when the set operation period is over.

15. The method of claim 13, wherein the controlled delivery device further comprises a syringe configured to deliver the quantity of the first agent into the aerosolizer.

16. The method of claim 15, wherein the controlled delivery device further comprises a tubing and one or more connectors providing a fluid path between an outlet of the syringe to an opening of the medication cup, the tubing is configured to deliver the first agent from the syringe onto the vibrating mesh pad of the aerosolizer.

17. The method of claim 16, wherein the controlled delivery device further comprises a syringe pump configured to deliver the first agent onto the vibrating mesh pad of the aerosolizer at a predetermined delivery rate.

18. The method of claim 17, wherein the controlled delivery device is configured to continuously operate the aerosolizer during the delivery of the first agent onto the vibrating mesh pad of the aerosolizer.

19. The method of claim 13, wherein the controlled delivery device is integrable with a humidifier or a ventilator via a T-joint connector.

20. A method of delivering an agent to a subject having COVID-19, the method comprising:
delivering a first agent to a subject for a treatment session comprising:
receiving a quantity of the first agent by an aerosolizer, the first agent comprising 3%-8.4% sodium bicarbonate at a pH ranging from 8.0 to 8.6 at 20° C.;
converting the first agent into aerosolized form, the aerosolized form of the first agent comprising droplets having an average size of 3 μm; and
delivering the first agent in the aerosolized form to the subject via a face mask configured to cover the mouth and nose of the subject;
wherein the quantity of the first agent is from 3 ml to 6 ml; and
wherein the treatment session has a treatment duration from 7 minutes to 30 minutes.

21. A kit for delivering an agent to a subject to prevent or relieve COVID-19 symptoms, the kit comprising:
a syringe;
an aerosolizer comprising a vibratory mesh pad;
a controller comprising a microprocessor;
a face mask configured to cover the mouth and nose of a subject;
an agent to be delivered to the subject, the agent comprising 3%-8.4% sodium bicarbonate at a pH ranging from 8.0 to 8.6 at 20° C.

22. A kit for delivering an agent to a subject who wants to prevent the onset of COVID-19 symptoms by taking the agent as a prophylactic, the kit comprising:
a pressurized canister having a valve and an opening, the pressurized canister containing an agent, the agent comprising 3%-8.4% sodium bicarbonate at a pH ranging from 8.0 to 8.6 at 20° C.; and
a face mask removably or fixedly attached to the pressurized canister at the opening, the face mask being configured to cover the nose and mouth of a subject;
wherein upon actuation of the valve, the agent exits the pressurized canister from the opening in aerosolized form into the face mask.
